(19) United States
(12) Patent Application Publication
(10) Pub. No.: US 2016/0250290 A1
(43) Pub. Date: Sep. 1, 2016

(54) GENE THERAPY FOR DIABETIC ISCHEMIC DISEASE

(71) Applicant: AnGes MG, Inc., Osaka (JP)

(72) Inventors: Ryuichi Morishita, Osaka (JP); Toshio Ogihara, Osaka (JP)

(73) Assignee: AnGes MG, Inc., Osaka (JP)

(21) Appl. No.: 14/867,866

(22) Filed: Sep. 28, 2015

Related U.S. Application Data
(63) Continuation of application No. 12/938,128, filed on Nov. 2, 2010, now abandoned.
Application No. 12/938,128 is a continuation of application No. 12/080,715, filed on Apr. 4, 2008, now abandoned.
Application No. 12/080,715 is a continuation of application No. 09/869,475, filed on Oct. 10, 2001, now abandoned.

(30) Foreign Application Priority Data
Oct. 29, 1999 (JP) 11309984

(51) Int. Cl. A61K 38/18; C12N 7/00; A61K 48/00

(52) U.S. Cl. CPC A61K 38/1833; A61K 48/0025; A61K 48/0075; A61K 48/0016; C12N 7/00; C12N 2760/18843

(57) ABSTRACT

It is possible to stimulate the angiogenesis of an ischemic site declined by diabetes and to recuperate ischemic disease by administering HGF (hepatocyte growth factor) gene to the diabetic ischemic.

### CROSS REFERENCE TO RELATED APPLICATIONS

This is a continuation of U.S. patent application Ser. No. 12/938,128, filed Nov. 2, 2010, which is a continuation of U.S. patent application Ser. No. 12/080,715, filed Apr. 4, 2008, which is a continuation of U.S. patent application Ser. No. 09/869,475, filed Oct. 10, 2001, which is the U.S. National Stage of PCT Application No. PCT/JP00/07502, filed Oct. 26, 2000, which in turn claims priority to Japanese Patent Application No. 11/309984, filed Oct. 29, 1999. All applications are incorporated herein in their entirety by reference.

### TECHNICAL FIELD

The present invention relates to a gene therapy agent and gene therapy method for diabetic ischemic disease utilizing a hepatocyte growth factor (HGF) gene. More specifically, the present invention relates to a method of gene therapy for diabetic ischemic disease which comprises the noninvasive administration of therapeutic agents of diabetic ischemic disease comprising an HGF gene as the effective ingredient or HGF gene.

### BACKGROUND ART

HGF is a protein that was first discovered as a strong growth factor for mature hepatocytes and the gene encoding it has been cloned (Biochem.Biophys.Res.Commun. 122, 1450 (1984); Proc. Natl. Acad. Sci. USA 83, 6489 (1986); FEBS Letter 22, 231 (1987); Nature 342, 440 (1989); Proc.Natl.Acad.Sci.USA 87, 3200 (1991)). Afterwards, according to researches, it has been revealed that HGF does not only work for repair and regeneration of the damaged liver, as a hepatocyte regeneration factor in vivo, but also has an angiogenic function and plays an important role in treatment and prevention of ischemic disease and artery disease (Symp.Soc.Exp.Biol. 47 cell behavior, 227-234 (1993); Proc. Natl. Acad. Sci. USA 90, 1937-1941 (19 93); Circulation 97, 381-390 (1998)). That is, it has been reported that upon administration of HGF to the rabbit lower limb ischemic model, significant angiogenesis is observed and improvement in blood flow, repression of blood pressure decrease and improvement in ischemic symptoms take place. According to these reports, it is believed today that HGF expresses and functions as one of the angiogenic factors.

As stated above, HGF has various functions to begin with functions as angiogenic factor, and many attempts have been made to use it as a drug. However, the half life of HGF in blood arose as a problem. The half life of HGF is as short as about 10 minutes, making it difficult to maintain its concentration in blood. Thus, problems arose as to how to deliver effective levels of HGF to the affected site.

Generally it is common knowledge that protein preparations are mostly administered intravenously and concerning the case above of HGF administration for the ischemic disease model, examples of intravenous and intra-arterial administration are shown (Circulation 97, 381-390 (1998)). In spite of the fact that effectiveness against ischemic disease or artery disease of intravenous or intra-arterial HGF administration to such animal models are revealed, specific administration methods, doses and so on effective for HGF are still under investigation. Particular effective administration methods or doses and such for HGF proteins are still to be determined, due to problems concerning its half life and delivery to the affected site described above.

On the other hand, the rapid progress lately in molecular biology has made it possible to activate cellular function by gene transfer methods and various attempts have been made. Some trials have been made for gene therapy of the heart region. There are some methods, like the coronary diffusional infusion method and such, reported for gene transfer methods but there is no case of gene transfer methods to the ischemic site, particularly intramuscular infusion method to the skeletal muscle showing effects on specific diabetic ischemic disease.

Further, it is known that angiogenesis hardly occurs and prognosis is unfavorable in ischemic disease complicated with or caused by diabetes. At present, it is not known whether HGF gene administration to such diabetic ischemic disease is effective or not.

### DISCLOSURE OF THE INVENTION

The object of this invention is to provide therapeutic agents and treatment methods for diabetic ischemic disease that utilize the HGF gene.

Inventors investigated to find out whether the HGF gene can be adapted to diabetic ischemic disease and revealed that extremely effective results are obtained by administering HGF gene directly to the ischemic affected site. Specifically, relating to lower limb ischemic disease, it was found out that effective results are obtained by administering HGF gene to the lower limb layer. As mentioned above, it is known that angiogenesis hardly occurs and prognosis is unfavorable in ischemic disease complicated with or caused by diabetes. Therefore, unlike mere ischemic disease, it had been unknown whether the HGF gene is effective toward diabetic ischemic disease. This invention revealed the effectiveness of the HGF gene for diabetic ischemic disease for the first time.

Since this method is a non-invasive treatment, it has the advantage that it is possible to administer the present gene repeatedly according to the condition.

Thus, the outline of the present invention is as follows:

(1) a therapeutic agent for diabetic ischemic disease, which comprises hepatocyte growth factor (HGF) as the effective ingredient;
(2) the therapeutic agent according to (1), used for administration to the ischemic site;
(3) the therapeutic agent according to (1) or (2), wherein the diabetic ischemic disease is selected from the group consisting of diabetic lower limb ischemic disease, diabetic ischemic neuropathy or diabetic ischemic myocardial infarction;
(4) the therapeutic agent according to (3), wherein the diabetic ischemic disease is diabetic lower limb ischemic disease;
(5) the therapeutic agent according to any of (1) to (4), used for administration into the muscle of the ischemic site;
(6) the therapeutic agent according to any of (1) to (5), wherein the HGF gene is in the form of a Sendai virus (HVJ)-liposome;
(7) the therapeutic agent according to any of (1) to (6), which is to be administered repeatedly as needed;
(8) the therapeutic agent according to any of (1) to (7), wherein the amount of HGF gene used is at least 50 μg;
(9) a method for the treatment of diabetic ischemic disease, which comprises the transfer of the HGF gene into human;
(10) the method according to (9), wherein the HGF gene is administered to an ischemic site;
(11) the method according to (9) or (10), wherein the diabetic ischemic disease is selected from the group consisting of diabetic lower limb ischemic disease, diabetic ischemic neuropathy or diabetic ischemic myocardial infarction;
(12) the method according to (11), wherein the diabetic ischemic disease is diabetic lower limb ischemic disease;
(13) the method according to any of (9) to (12), wherein the HGF gene is administered into the muscle of ischemic site;
(14) the method according to any of (9) to (13), wherein the HGF gene is in the form of a Sendai virus (HVJ)-liposome;
(15) the method according to any of (9) to (14), wherein the HGF gene is administered repeatedly as needed;
(16) the method according to any of (9) to (15), wherein the amount of HGF gene to be administered is at least 50 μg;
(17) use of the HGF gene for preparing therapeutic agents for diabetic ischemic disease;
(18) the use according to (17), wherein the diabetic ischemic disease is selected from the group consisting of diabetic lower limb ischemic disease, diabetic ischemic neuropathy or diabetic ischemic myocardial infarction;
(19) the use according to (18), wherein the diabetic ischemic disease is diabetic lower limb ischemic disease;
(20) the use according to any of (17) to (19), wherein the HGF gene is in the form of a Sendai virus (HVJ)-liposome; and
(21) the use according to any of (17) to (20), wherein the amount of HGF gene to be used is at least 50 μg.

### BRIEF DESCRIPTION OF THE DRAWINGS

FIG. 1 is a graph showing changes in blood perfusion ratio over time of the group of rats with diabetic lower limb ischemia in reference 1 and of the control group, in which lower limb ischemia was induced in normal rats.

FIG. 2 is a graph showing the internal HGF concentration in ischemic muscle of the group of rats with diabetic lower limb ischemia in reference 1 and of the control group, in which lower limb ischemia was induced in normal rats.

FIG. 3 shows the blood perfusion ratio of the group of rats with diabetic lower limb ischemia in Experiment 1, to which HGF gene was administered or not, and of the control group, in which lower limb ischemia was induced in normal rats.

FIG. 4 is a graph showing the result of a comparison of the number of blood vessels of the group of rats with diabetic lower limb ischemia in Experiment 2, to which HGF gene was administered or not, and of the control group, in which lower limb ischemia was induced in normal rats by ALP (alkaline phosphatase) staining of the skeletal muscle of the lower limb ischemic site.

FIG. 5 is a graph showing the MMP-1 concentration in the culture supernatant of the glucose added angioendothelial cell in reference 2, to which HGF was added or not, and of the control group, to which no glucose was added.

FIG. 6 is a graph showing the amount of mRNA of transcription factor which is expressed in the angioendothelial cell, of the group of glucose added angioendothelial cell in reference 3, to which HGF was added or not, and of the control group to which no glucose was added.

### BEST MODE FOR CARRYING OUT THE INVENTION

As used herein “HGF gene” means a gene that can express HGF (the HGF protein). Specifically, cDNA of HGF described in Nature 342: 440 (1989); Japanese Patent Publication No., 2777678; Biochem.Biophys.Res.Commun. 163: 967 (1989); and Biochem.Biophys.Res.Commun. 172: 321 (1990) and so on integrated into suitable expression vectors (non-viral vector, viral vector) described below are to be mentioned. The base sequence of the cDNA encoding HGF gene of the present invention has been described in the above literature and is also registered with databases such as GENBANK. Thus, based on such sequence information, using a suitable DNA portion as a PCR primer, it is possible to clone the cDNA of HGF, for example, by performing a RT-PCR reaction on mRNA derived from the liver or leukocytes. Such cloning can easily be performed by a person skilled in the art according to a basic textbook, such as Molecular Cloning 2nd Ed., Cold Spring Harbor Laboratory Press (1989).

The HGF genes of the present invention are not restricted to the above mentioned genes but also include those genes that express proteins with substantially the same function as HGF. That is, the following genes fall under the category of the HGF gene of the present invention: 1) DNA that hybridize to said cDNA under stringent conditions, and 2) DNA encoding proteins having amino acid sequence in which 1 or more (preferably a few) amino acids are substituted, deleted and/or added to the protein encoded by said cDNA and which encodes proteins having the function as HGF. Above DNAs of 1) and 2) can be readily obtained, for example, by site-directed mutagenesis, PCR method, ordinal hybridization method and so on. Such methods can be easily accomplished according to the above basic textbook.

Subsequently, methods of gene transfer, dosage forms, doses and the like for use in gene therapy of the present invention are explained.

The dosage form of a gene therapy agent comprising the above gene as the effective ingredient to be administered to patients are roughly classified into two groups: one is the case in which a nonviral vector is used, and the other is in which a viral vector is used. Methods for preparation and administration thereof are explained in detail in experimental manuals (Supplement of Experimental Medicine, Basic Technology in gene therapy, Yodosha (1996); Supplement of Experimental Medicine, Experimental Methods in Gene Introduction and Expression Analysis, Yodosha (1997); Handbook for Development and Research of Gene Therapy, Japan Society of Gene Therapy ed., NTS (1999)). Specifics are explained below.

A. Usage of a nonviral vector

Using a recombinant expression vector in which the gene of interest has been integrated into a commonly used gene expression vector, may be used to introduce the gene of interest into cells or tissue by the following method etc.

Illustrative methods of gene transfer into cells include the lipofection method, calcium phosphate co-precipitation method, DEAE-dextran method, direct DNA introduction methods using micro glass tubes, and the like.

Regarding methods of gene transfer into the tissue, a recombinant expression vector may be incorporated into the cell by subjecting it to any of the method, such as the method of gene transfer with internal type liposome, method of gene introduction with electrostatic type liposome, HVJ-liposome method, improved HVJ-liposome method (HVJ-AVE liposome method), receptor-mediated gene introduction method, method of introducing DNA molecules together with carriers (metal particles) by a particle gun, method of directly introducing naked-DNA, method of introduction with positively-charged polymers and the like.

Among them, HVJ-liposome is a fusion product prepared by enclosing DNA into liposome made of lipid bilayer, which is fused to inactivated Sendai virus (Hemagglutinating virus of Japan: HVJ). The HVJ-liposome method is characterized by a very high fusing activity with the cell membrane as compared to the conventional liposome method, and is a preferred mode of introduction. For the method of preparing HVJ-liposome, see the literature for details (Separate volume of; Experimental Medicine, Basic Technology in gene therapy, Yodosha (1996); experimental Methods in Gene Introduction and Expression Analysis, Yodosha (1997); J. Clin. Invest. 93:1458-1464(1994); Am. J. Physiol. 271: R1212-1220 (1996)) and the like, and experimental examples described below for details. The HVJ-liposome method is exemplified by the method as described in, for example, Molecular Medicine, 30, 1440-1448 (1993), Experimental Medicine, 12, 1822-1826 (1994), and Protein, Nucleic Acid, Enzyme, 42, 1806-1813 (1997), preferably that described in Circulation, 92 (Suppl. II), 479-482 (1995). In particular, the Z strain (available from ATCC) is preferred as the HVJ strain, but other HVJ strains (for example, ATCC VR-907 and ATCC VR-105) may also be used.

Furthermore, the method of directly introducing naked-DNA is the most simple method among the methods described above, and in this regard a preferred method of introduction.

Expression vectors as used herein may be any expression vectors so long as they permit the in vivo expression of the gene of interest. Examples include expression vectors such as pCAGGS (Gene 108:193-200 (1991)), pBK-CMV, pcDNA3.1, pZeoSV (INVITROGEN, STRATAGENE) and the like.

B. Usage of a viral vector

Representative methods that use viral vectors include those using viral vectors such as recombinant adenovirus, retrovirus and the like. More specifically, the gene of interest can be introduced into a DNA or RNA virus such as detoxified retrovirus, adenovirus, adeno-associated virus, herpes virus, vaccinia virus, poxvirus, poliovirus, Sindbis virus, Sendai virus, SV40, human immunodeficiency virus (HIV) and the like, which is then infected to the cell to introduce the gene into the cell.

Among the above viral vectors, the efficiency of infection of adenovirus is known to be much higher than that of other viral vectors. In this regard, it is preferred to use an adenovirus vector system.

As methods of introducing a gene therapy agent into a patient, there are in vivo methods, which permit direct introduction of the gene therapy agent into the body, and ex vivo methods, in which certain cells are removed from a human, wherein the gene therapy agent is introduced and which are then returned into the body (Nikkei Science, April 1994 issue pp.20-24; Monthly Yakuji, 36(1):23-48 (1994); Supplement To Experimental Medicine 12(15) (1994); Handbook for Development and Research of Gene Therapy, NTS (1999)). According to the present invention, the in vivo method is preferred.

Dosage forms may take various forms according to various administration regimens described above (for example, liquids). When, for example, an injection containing the gene as the effective ingredient is to be used, said injection may be prepared by dissolving the effective ingredients into a standard solvent (a buffer such as PBS, physiological saline, sterile water, etc.). The injection liquid may then be filter-sterilized with filter as needed, and then filled into sterilized containers. Conventional carriers and so on may be added to the injection. Liposomes, such as HVJ-liposome may take the form of suspensions, frozen formulations, centrifugation-concentrated frozen formulations and the like.

It is possible to use known factors having angiogenic functions, additionally or alone besides the HGF gene used in this invention. For example, it is reported that factors such as VEGF and FGF have an angiogenic function and therefore such genes can be used. Further, growth factors such as EGF are reported to repair cell damage in various tissues and thus genes encoding them can be also used.

The diabetic ischemic disease herein includes diseases such as diabetic lower limb ischemic disease, diabetic ischemic neuropathy and diabetic ischemic cardiac infarction and so on, and the therapeutic agent of this invention can be applied to any of these diseases. Moreover, the therapeutic agent of this invention can be applied not only to patients with critical diabetic ischemic disease but also to patients with progressively mild symptoms.

Proper methods and sites for administration adequate for the disease or symptom to be treated are selected for the gene therapy agent of this invention. As to the administration methods, parenteral administration methods are preferred. As a preferable administration site, the ischemic site can be mentioned. “Ischemic site” herein refers to the site including the affected site of ischemia and surrounding sites thereof.

Specifically, it is possible to administer into the blood vessel or into the muscle of the ischemic site. However, administration into the muscle of the ischemic site is preferred. In other words, administration into the skeletal muscle of the lower limb ischemic site enables stimulation of angiogenesis in the affected site of ischemia and improvement of blood flow. Thereby, it enables recovery and normalization of the function of the ischemic site. While in cardiopathy, such as cardiac infarction, it is possible to gain similar effect by administering into the cardiac muscle.

Examples of preferred administration methods include, for example, administration by noninvasive catheter, noninvasive injector and so on. Moreover, administration methods which utilize a noninvasive catheter, noninvasive injector and such under the usage of echo can be mentioned. As a method using noninvasive catheter, for example, injecting HGF genes directly into the cardiac muscle from the ventricle inner space in a cardiopathy can be indicated.

Application of the HGF gene of the present invention makes positive treatment for patients with diabetic ischemic disease possible. For example, it enables the recovery of function inpatients with critical symptoms to whom no option, other than surgical excision of the affected site, is left.

Dosage of the therapeutic agent of this invention varies depending on the symptoms of the patient but HGF genes about 1 μg to about 50 mg, preferably about 10 μg to about 5 mg, more preferably about 50 μg to about 5 mg per adult patients can be defined.

The therapeutic agent of this invention is suited for administration once every few days or once every few weeks and the frequency of administration is appropriately selected depending on symptoms of patients. According to the therapeutic treatment of the invention, genes are administered noninvasively and, therefore, desired genes can be administered as much as the condition demands.

The present invention will now be specifically explained with reference to the following examples. It should be noted, however, that the present invention is not limited by these examples in any way.

Materials and Methods

Preparation of HVJ-liposome agent

10 mg dried lipid (a 1: 4. 8: 2 mixture of phosphatidyl serine, phosphatidyl choline and cholesterol) and 200 μl balanced salt solution (137 μM NaCl, 5.4 (μM KCl, 10 μM Tris-HCl; pH7. 6) containing HGF gene (100 μg)-HMGl (high mobility group 1 nuclear protein, 25 μg) was mixed and, by stirring vigorously with ultrasonication, liposomes were formed. Purified Sendai virus (Z strain) was irradiated with UV (110 erg/mm²/sec) for 3 minutes. Liposome suspension was mixed with Sendai virus (HVJ), heated at 4° C. for 10 minutes, and then heated at 37° C. for 30 minutes. Free HVJ was discarded and thus obtained HVJ liposome agent.

Experimental Animals

Administered group: ischemic rat of diabetic rat (DM-rat)

Control rat: ischemic rat of normal rat

Administration method of HVJ-liposome agent

By surgically excising the femoral artery of one leg of the diabetic rat (16 weeks old; 6 animals per group), to which diabetes was provoked by interperitoneal administration of streptozotocin, ischemic state in the lower limb site was produced.

HVJ-liposome preparation containing HGF gene was injected to the lower limb skeletal muscle.

Details of examination

After administration of the liposome preparation, the blood flow of the lower limb was measured by laser Doppler imager (LDI) using laser scatter analysis as the index of bypass circulation formation and effects of improvement in blood flow. The average of colored histogram of ischemic lower limb to that of the normal lower limb was taken as the perfusion ratio.

The density of blood capillary in the lower limb ischemic site was measured by alkaline phosphatase (ALP) staining, and the result of diabetic lower limb ischemic rat group was compared to that of the control lower limb ischemic rat group. Alternatively, comparison between the groups to which HGF gene was administered and to which no HGF gene was administered was made.

Reference 1

HGF expression state in the lower limb ischemic site of the diabetic rat

Ischemic state in the lower limb site was produced by surgical excision of the femoral artery of one leg of the diabetic rats (16 weeks old; 6 animals per group), to which diabetes was provoked by interperitoneal administration of streptozotocin, and of normal rats (16 weeks old; 6 animals per group) as the control group.

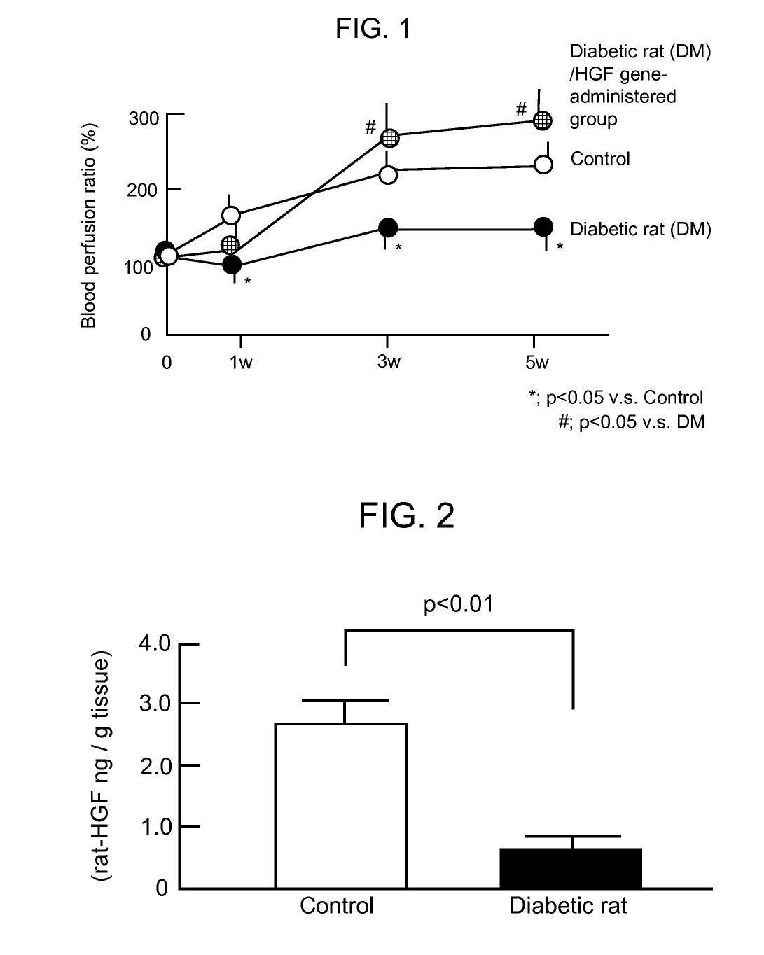

After one week, the perfusion ratio of the ischemic site was measured by laser Doppler imager. The perfusion ratio of the ischemic site or the diabetic lower limb ischemic rat was much lower than that of the control lower limb ischemic rat (see FIG. 1).

The perfusion ratio of the lower limb was measured again 3 weeks and 5 weeks later, and the same results were obtained. That is, the perfusion ratio of the lower limb of the diabetic lower limb ischemic rat was much lower than that of the control lower limb rat (see FIG. 1).

The internal HGF concentration in the muscles was significantly lower in the muscles of the ischemic site of the diabetic lower limb ischemic rat than that of the control lower limb ischemic rat. This result indicates that angiogenesis in diabetes is poor due to the decrease of internal HGF in the muscles. Therefore, angiogenesis hardly occurs in diabetic lower limb ischemic rat and bypass circulation does not develop (see FIG. 2).

Experiment 1

Effect of HGF gene therapy against diabetic lower limb ischemic rat (I)

Ischemic state in the lower limb site was produced by surgical excision of the femoral artery of one leg of the normal rats (16 weeks old; 6 animals per group) and that of diabetic rats (16 weeks old; 6 animals per group), to which diabetes was provoked by interperitoneal administration of streptozotocin. After surgical excision of the femoral artery, infusion of HVJ-liposome preparation containing HGF gene (50 μg) was injected into the muscle of the lower limb ischemic site.

After 3 weeks, the perfusion ratio of the ischemic site was measured by laser Doppler imager. The perfusion ratio of the ischemic site of the diabetic lower limb ischemic rat, to which HGF gene was administered, showed significant increase compared to that of the control lower limb ischemic rat or that of the diabetic lower limb ischemic rat above with no administration.

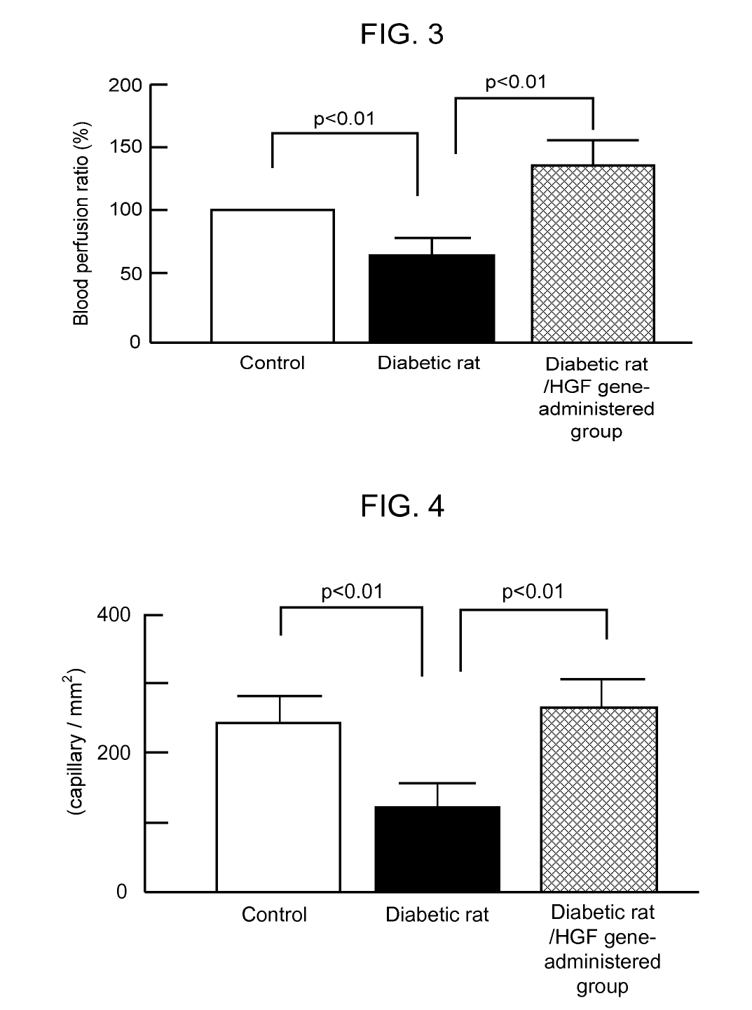

Taking perfusion ration of the control lower limb ischemic rat as 100%, that of the HGF gene untreated diabetic lower limb ischemic rat was 67% and that of the HGF administered diabetic lower limb ischemic rat was 129%. The results are shown in FIG. 3 (see FIG. 1).

Experiment 2

Effect of HGF gene therapy against diabetic lower limb ischemic rat (II)

Diabetic lower limb ischemic rat and control lower limb ischemic rat treated as above were prepared and subjected to HGF gene therapy. 5 weeks later, the skeletal muscle of the lower limb ischemic site was taken from each animal, subjected to ALP staining and the blood vessel count per unit area was compared. The blood vessel count of HGF gene untreated diabetic lower limb ischemic rat was significantly smaller as that of the control lower limb ischemic rat, and that of the HGF administered diabetic lower limb ischemic rat was significantly increased. The results are shown in FIG. 4.

Reference 2

Influence of glucose concentration and HGF addition against MMP-1 production of the angioendothelial cell

The angioendothelial cells (derived from human aorta) were cultured in three types of serum free medium containing glucose at a concentration of 0, 25 mM and 50 mM, respectively. After 24 hours of cultivation, the MMP-1 concentration in the supernatant of the culture media was measured.

Each sample was compared to that in which 100 ng/ml of HGF was added 30 minutes before glucose addition.

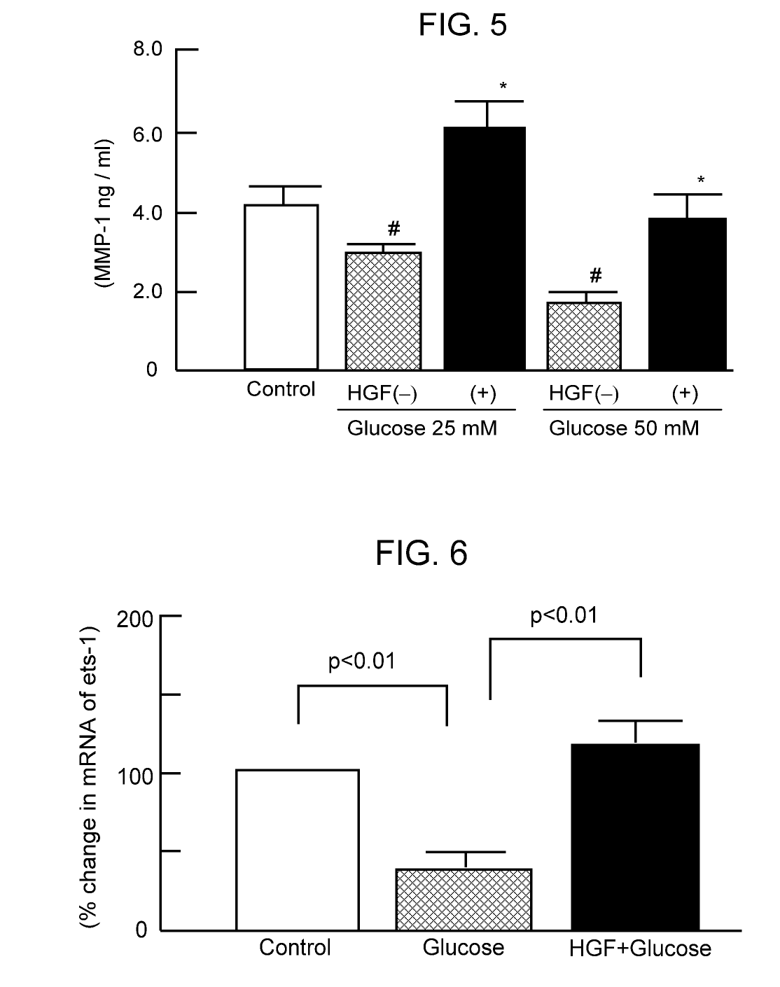

The MMP-1 concentration of the supernatant decreased significantly depending on the glucose concentration, and it was shown that HGF treatment inhibited the decrease of MMP-1 and, in turn, induced its increase. The results are shown in FIG. 5.

Reference 3

Effect of HGF against angioendothelial cells (Changes of transcription factor ET5-1 related to angiogenesis)

Cultivation of angioendothelial cell was conducted as in reference 1, and expression of mRNA of the transcription factor ETS-1 in the cell was detected. Taking the mRNA of ETS-1 in the control endothelial cell as 100%, that of the HGF untreated angioendothelial cell decreased in a glucose dependent manner. On the other hand, HGF treated angioendothelial cells expressed the mRNA of ETS-1 at the same or more level compared to that of the control group (P<0.01). The results are shown in FIG. 6.

As described above, angioendothelial cells under high glucose concentration show a decrease in MMP-1 expression, which is a matrix cleaving enzyme essential for angiogenesis, and show a decrease in the expression of mRNA of the transcription factor ETS-1, which is expressed and increases during angiogenesis.

Consequently, it was revealed that angiogenesis hardly occurs under high glucose condition. On the other hand, it was shown that by adding HGF to the angioendothelial cell under high glucose condition, MMP-1 expression and mRNA of ETS-1 expression increases significantly. Thus, it was revealed that HGF makes angiogenesis easier.

### Industrial Applicability

The therapeutic agent for diabetic ischemic disease containing an HGF gene as the effective ingredient improves poor angiogenesis specific to the affected site of diabetic ischemia with decrease in HGF expression and shows significant angiogenic effect. Therefore, it enables the improvement of the condition by increasing the blood flow in the affected site of ischemia. Moreover, the therapeutic agent of this invention can be administered once or more, depending on the symptoms of the patient, thereby stimulating angiogenesis. Therefore, according to these effects, the therapeutic agent of this invention makes it possible to treat diabetic lower limb ischemic disease, diabetic ischemic neuropathy and diabetic cardiac infarction.

1. A method for the treatment of diabetic ischemic disease in a subject, comprising nonivasively administering a therapeutically effective amount of a hepatocyte growth factor (HGF) gene directly to a muscle of an ischemic site, and measuring blood flow, wherein expression of the HGF gene increases blood flow in the diabetic subject compared to the blood flow of an HGF-treated non-diabetic subject with ischemic disease, thereby treating the diabetic ischemic disease.

2. The method according to claim 1, wherein the HGF gene is in the form of a Sendai virus (HVJ)-liposome.

3. The method according to claim 1, wherein about 50 μg to about 5 mg the HGF gene is administered to the subject, thereby treating the diabetic ischemic disease.

4. The method of claim 1, wherein the HGF gene is administered as a naked DNA.

5. The method of claim 1, wherein the HGF gene is administered as a naked expression vector DNA.

6. The method of claim 1, wherein administration of the HGF gene increases blood flow compared to the blood flow following administration of an HGF-recombinant protein.

7. The method of claim 1, wherein the diabetic ischemic disease is selected from the group consisting of diabetic lower limb ischemic disease, diabetic ischemic neuropathy, and diabetic ischemic myocardial infarction.

8. The method of claim 7, wherein the diabetic ischemic disease is diabetic lower limb ischemic disease.
